(19) United States
(12) Patent Application Publication
(10) Pub. No.: US 2004/0054174 A1
(43) Pub. Date: Mar. 18, 2004

(54) Nile-red luminescent compound, process for producing the same, and luminiscent element utilizing the same

(76) Inventors: Tadao Nakaya, Tokyo (JP); Takao Yamauchi, Kanagawa (JP); Tomoyuki Saikawa, Kanagawa (JP); Akio Tajima, Kanagawa (JP); Hidemasa Mori, Kanagawa (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/416,586

(22) PCT Filed: Jun. 29, 2001
(86) PCT No.: PCT/JP01/05671

(30) Foreign Application Priority Data
Aug. 4, 2000 (JP) 2000-236670
Nov. 15, 2000 (JP) 2000-348516

(51) Int. Cl. C07D 265/34

(52) U.S. Cl. 544/99

(57) ABSTRACT

The objectives of this invention are to provide a novel Nile Red luminescent compound capable of emitting a light of which color is nearly crimson, a novel process for producing the same and a luminescent compound capable of emitting at a high luminance a nearly crimson light. This invention relates to a Nile Red luminescent compound emitting red light made by changing C═O in a Nile Red compound to C═CH—Ar, wherein Ar is an aromatic group including fluorine atoms, and by introducing an electron attractive aromatic acetonitrile into the skeleton of the Nile Red compound; a novel process for producing the Nile Red luminescent compound that comprises reacting a Nile Red pigment with an electron attractive aromatic acetonitrile, and a luminescent compound comprising a composition that includes the Nile Red luminescent compound.

### TECHNICAL FIELD

The present invention relates to a Nile Red luminescent compound emitting red light, a process for producing the same and a luminescent element utilizing the same. More particularly, this invention relates to a Nile Red luminescent compound capable of emitting at a high luminance a light of which color is nearly crimson upon the application of electric energy, a novel process of producing the compound and a luminescent element utilizing the same.

### BACKGROUND ART

For organic electroluminescent elements, which are often abbreviated to “organic EL elements”, have been proposed various organic compounds.

However, compounds that are capable of emitting red light at a high luminance and endurable against heat, light, etc. have not been developed.

The objective of this invention is to provide an organic compound capable of emitting red light at a high luminance, and/or capable of emitting a light of which color is such a red as the value on the x-axis in the CIE chromaticity is over 0.63, and further endurable against heat, light, etc. This invention also aims for providing a novel process for producing the organic compound and a luminescent element utilizing the compound.

### SUMMARY OF THE INVENTION

In order to solve the aforementioned problems, this invention provides a Nile Red luminescent compound emitting red light that has a structure represented by the following general formula (1):

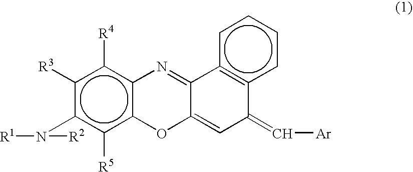

wherein R¹ is a lower alkyl having 1-5 carbon atoms, or forms —CH₂CH₂—CR⁶R⁷-together with R³ (wherein the carbon atom of —CR⁶R⁷— moiety is bound to the benzene moiety of chemical formula (1), each of R⁶ and R⁷ is hydrogen atom or a lower alkyl having 1-5 carbon atoms, and R⁶ and R⁷ may be the same or different from each other); R² is a lower alkyl having 1-5 carbon atoms, or forms —CH₂CH₂—CR⁸R⁹— together with R⁵ (wherein the carbon atom of —CR⁸R⁹— moiety is bound to the benzene moiety of chemical formula (1), each of R⁸ and R⁹ is hydrogen atom or a lower alkyl having 1-5 carbon atoms, and R⁸ and R⁹ may be the same or different from each other); R³ is hydrogen atom, forms —CH₂CH₂—CR⁶R⁷— with R¹, or forms with R⁴ a naphthalene ring including as apart thereof the benzene moiety of chemical formula (1); R⁴ is hydrogen atom, or forms with R³ a naphthalene ring including as a part thereof the benzene moiety of chemical formula (1); and R⁵ is hydrogen atom, or forms —CH₂CH₂—CR⁸R⁹— with R².

In the formula, Ar means one of the following general formulae (2), (4) and (5):

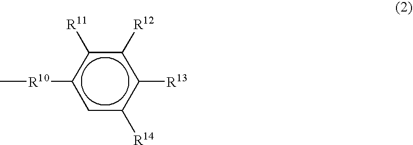

wherein R¹⁰ is a single chemical bond or methylene group; R¹¹ is hydrogen atom, or forms —CF₂—O—CF₂— with R¹²; R¹² is fluorine atom, cyano group or a lower alkyl having 1-5 carbon atoms and at least one fluorine atom, forms —CF₂—O—CF₂— with R¹¹, or forms —CF₂—O—CF₂— with R¹³; R¹³ is hydrogen atom, cyano group, fluorine atom or a lower alkyl having 1-5 carbon atoms and at least one fluorine atom, forms —CF₂—O—CF₂— with R¹², or is a group represented by general formula (3); and R¹⁴ is hydrogen atom or a lower alkyl having 1-5 carbon atoms and at least one fluorine atom when R¹³ is hydrogen atom, and R¹⁴ is hydrogen atom when R¹³ is not hydrogen atom.

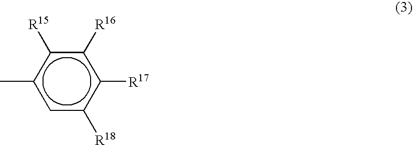

wherein R¹⁵ is hydrogen atom, or forms —CF₂—O—CF₂— with R¹⁶; R¹⁶ is fluorine atom, cyano group or a lower alkyl having 1-5 carbon atoms and at least one fluorine atom, forms —CF₂—O—CF₂— with R¹⁵, or forms —CF₂—O—CF₂— with R¹⁷; R¹⁷ is hydrogen atom, cyano group, fluorine atom or a lower alkyl having 1-5 carbon atoms and at least one fluorine atom, or forms —CF₂—O—CF₂— with R⁶; and R¹ is hydrogen atom or a lower alkyl having 1-5 carbon atoms and at least one fluorine atom when R¹⁷ is hydrogen atom, and R¹⁸ is hydrogen atom when R¹⁷ is not hydrogen atom.

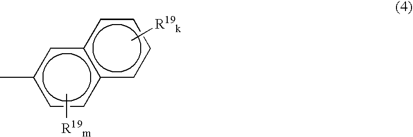

wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other.

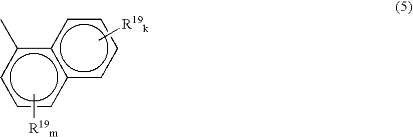

wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other.

As another solution to the aforementioned problems, this invention provides a process for producing the Nile Red luminescent compound emitting red light that has the structure represented by aforementioned general formula (1), which comprises reacting a Nile Red pigment represented by general formula (6) with an electron attractive aromatic acetonitrile represented by general formula (7).

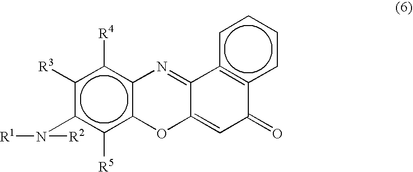

wherein R¹, R², R³, R⁴ and R⁵ are respectively the same as those defined in the explanation of formula (1).

NC—CH₂—Ar (7)

wherein Ar is the same as that defined in the explanation of formula (1).

As further means to solve the aforementioned problems, this invention provides a luminescent element comprising a pair of electrodes and a luminescent layer including the Nile Red luminescent compound emitting red light represented by formula (1) between the electrodes.

In a preferred embodiment of the invention relating to the luminescent element, the element further comprises a hole-transporting layer between the luminescent layer and the cathode, which is one of the electrodes.

In another preferred embodiment of this invention relating to the luminescent element, the luminescent layer includes the Nile Red luminescent compound emitting red light and a host pigment.

In a further preferred embodiment of this invention relating to the luminescent element, the luminescent layer and the hole-transporting layer are formed by vapor deposition.

In a still further embodiment of this invention relating to the luminescent element, the luminescent layer includes the Nile Red luminescent compound emitting red light, an electron-transporting substance and a hole-transporting high polymer.

In another preferred embodiment of this invention, the luminescent layer is formed through the application of the layer.

In still another preferred embodiment of this invention, the luminescent layer further includes at least one selected from the group consisting of a diphenylvinyl biphenol compound emitting blue light and a stilbene compound emitting blue light, and at least one selected from the group consisting of a coumarin compound emitting green light, an indophenol compound emitting green light and an indigo compound emitting green light.

### BRIEF DESCRIPTION OF THE DRAWINGS

FIG. 1 is a ¹H-NMR chart of Nile Red luminescent compound A in Example 1.

FIG. 2 is an IR chart of Nile Red luminescent compound A in Example 1.

FIG. 3 is an IR chart of Nile Red luminescent compound B in Example 2.

FIG. 4 is an NMR chart of the Nile Red luminescent compound in Example 16.

FIG. 5 is an NMR chart of the Nile Red luminescent compound in Example 17.

FIG. 6 is an NMR chart of the Nile Red luminescent compound in Example 10.

FIG. 7 is an NMR chart of the Nile Red luminescent compound in Example 18.

FIG. 8 is an NMR chart of the Nile Red luminescent compound in Example 19.

FIG. 9 is an NMR chart of 6-amino-3-(diisopropylamino)-2-nitrophenol in Example 20.

FIG. 10 is an NMR chart of the Nile Red compound derivative in Example 20.

FIG. 11 is an NMR chart of the Nile Red luminescent compound in Example 20.

### DETAILED DESCRIPTION OF THE PREFERRED EMBODIMENTS

The Nile Red luminescent compound in accordance with this invention is represented by general formula (1):

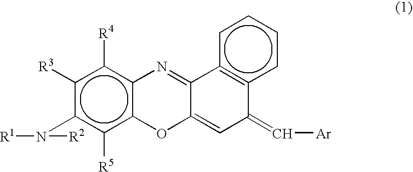

In this formula, R¹ is a lower alkyl group having 1-5 carbon atoms. The lower alkyl group includes methyl group, ethyl group, propyl group, butyl group and pentyl group.

R² is a lower alkyl group having 1-5 carbon atoms, which includes the same groups as R¹. R¹ and R² may be the same or different from each other.

Together with R³, R¹ forms —CH₂CH₂—CR⁶R⁷— (wherein the carbon atom of —CR⁶R⁷— moiety is bound to the benzene moiety of chemical formula (1), each of R⁶ and R⁷ is hydrogen atom or a lower alkyl having 1-5 carbon atoms, and R⁶ and R⁷ may be the same or different from each other).

When R¹ and R² are lower alkyl groups, preferable —NR¹R² includes diethylamino group, di-n-propylamino group, di-1-propylamino group, butyl group, etc.

Together with R⁵, R² forms —CH₂CH₂—CR⁸R⁹— (wherein the carbon atom of —CR⁸R⁹— moiety is bound to the benzene moiety of chemical formula (1), each of R⁸ and R⁹ is hydrogen atom or a lower alkyl having 1-5 carbon atoms, and R⁸ and R⁹ may be the same or different from each other).

When R¹ forms —CH₂CH₂—CR⁶R⁷— with R³, and R² forms —CH₂CH₂—CR⁸R⁹— with R⁵, general formula (1) becomes following general formula (8):

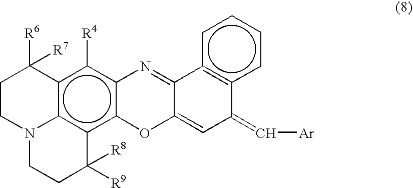

In this formula (8), R⁴, R⁶, R⁷, R⁸, R⁹ and Ar denote the same as those mentioned above.

Both R³ and R⁴ are hydrogen atoms, or form together a naphthalene ring including as a part thereof the benzene moiety of chemical formula (1). The red light-emitting luminescent compound that has the naphthalene ring including as a part thereof the benzene moiety formed by R³ and R⁴ is represented by general formula (9).

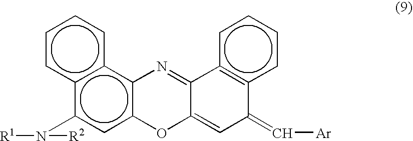

In formula (9), R¹, R² and Ar denote the same as those mentioned above.

In general formula (1), Ar has a structure represented by general formula (2), (4) or (5).

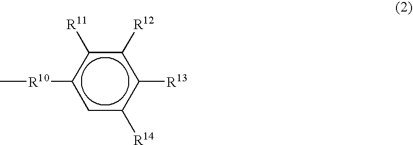

In this formula, R¹⁰ is a single chemical bond or methylene group.

R¹¹ is hydrogen atom, or forms —CF₂—O—CF₂— with R¹².

R¹² is fluorine atom, cyano group or a lower alkyl having 1-5 carbon atoms and at least one fluorine atom, forms —CF₂—O—CF₂— with R¹¹, or forms —CF₂—O—CF₂— with R¹³.

R¹³ is hydrogen atom, cyano group, fluorine atom or a lower alkyl having 1-5 carbon atoms and at least one fluorine atom, forms —CF₂—O—CF₂— with R¹², or is a group represented by general formula (3).

R¹⁴ is hydrogen atom or a lower alkyl that has 1-5 carbon atoms and at least one fluorine atom when R¹³ is hydrogen atom, and R¹⁴ is hydrogen atom when R¹³ is not hydrogen atom.

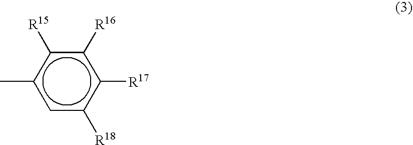

In this formula, R¹⁵ is hydrogen atom, or forms —CF₂—O—CF₂— with R¹⁶.

R¹⁶ is fluorine atom, cyano group or a lower alkyl having 1-5 carbon atoms and at least one fluorine atom, forms —CF₂—O—CF₂— with R¹⁵, or forms —CF₂—O—CF₂— with R¹⁷.

R¹⁷ is hydrogen atom, cyano group, fluorine atom or a lower alkyl having 1-5 carbon atoms and at least one fluorine atom, or forms —CF₂—O—CF₂— with R¹⁶.

R¹⁸ is hydrogen atom when R¹⁷ is not hydrogen atom, or a lower alkyl having 1-5 carbon atoms and at least one fluorine atom when R¹⁷ is hydrogen atom.

The lower alkyl having 1-5 carbon atoms and at least one fluorine atom represented by R¹², R¹³, R⁶ and R¹⁷ includes a methyl group including at least one fluorine atom such as trifluoromethyl group, difluoromethyl group and monofluoromethyl group, an ethyl group including at least one fluorine atom such as pentafluoroethyl group, a propyl group including at least one fluorine atom such as hexafluoropropyl group, a pentyl group including at least one fluorine atom, etc. Among them, preferable is the methyl group including at least one fluorine atom.

In Ar of general formula (1) or (9), when R¹⁰ is a single chemical bond or methylene group, examples of the preferable combination of R¹¹, R¹², R¹³ and R¹⁴ are shown in Table 1.

TABLE 1

| Combination | | | | |
|---|---|---|---|---|
| Examples | R¹¹ | R¹² | R¹³ | R¹⁴ |
| 1 | —H | —CF₃ | —H | —CF₃ |
| 2 | —H | —F | —CF₃ | —H |
| 3 | —H | —CF₃ | —F | —H |
| 4 | —H | —CF₃ | —CN | —H |
| 5 | —H | —CN | —CF₃ | —H |
| 6 | —H | —F | —CN | —H |
| 7 | —H | —CN | —F | —H |
| 8 | —H | —F | —CF₃ | —H |
| 9 | CF₂—O—CF₂— | | —H | —H |
| 10 | —H | —CF₂—O—CF₂— | | —H |
| 11 | —H | General | —H, —CF_{3,} | —H |
| | | formula (3) | —CN or —H | |
| 12 | —F | | —CF₃ | —H |
| 13 | —CF₃ | —H | —CF₃ | —H |

Other than the preferable examples shown in Table 1, also preferred examples of Ar are as follows: a fluorinated phenyl such as 2,4-difluorophenyl group, 2,5-difluorophenyl group, 2,6-difluorophenyl group, 3,4-difluorophenyl group, 3,5-difluorophenyl group, etc.; a trifluoromethylphenyl group such as 3-trifluoromethylphenyl group, 4-trifluoromethylphenyl group, 3,5-bis (trifluoromethyl)phenyl group, etc.; and a fluorotrifluoromethylphenyl group such as 4-fluoro-3-trifluoromethylpheyl group, 6-fluoro-2-trifluoromethylphenyl group, etc.

One of Ar's is shown by general formula (4):

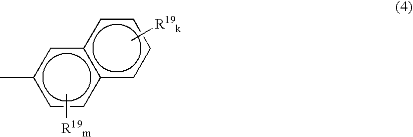

wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other.

Another one of Ar's is represented by general formula (5):

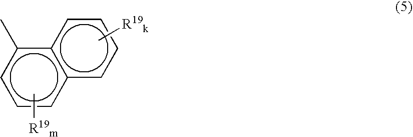

wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other.

Because the naphthyl group represented by formula (4) or (5) has electron attractive groups such as fluorine atom, cyano group and a lower alkyl having 1-5 carbon atoms and at least one fluorine atom, and therefore π electrons on the skeleton of the Nile Red compound and the fluorinated substituents or the cyano groups are super-conjugated, it facilitates the emission of red light.

The lower alkyl having 1-5 carbon atoms and at least one fluorine atom, bonded to the naphthyl group, is the same as that in general formula (2). Among them, trifluoromethyl group is preferable.

Among the naphthyl groups represented by general formula (4), 1-naphthyl groups include, for example: a (trifluoromethyl)-1-naphthyl group having a trifluoromethyl group at the 2-, 3-, 4-, 5-, 6-, 7- or 8-position; a fluoro-1-naphthyl group having a fluorine atom at the 2-, 3-, 4-, 5-, 6-, 7-or 8-position; a bis(trifluoromethyl)-1-naphthyl group having two trifluoromethyl groups at the 2- and 3-positions, the 2- and 4-positions, the 2- and 5-positions, the 2- and 6-positions, the 2- and 7-positions, the 2- and 8-positions, the 3- and 4-positions, the 3- and 5-positions, the 3- and 6-positions, the 3- and 7-positions, the 3- and 8-positions, the 4- and 5-positions, the 4- and 6-positions, the 4- and 7-positions, the 4- and 8-positions, the 5- and 6-positions, the 5- and 7-positions, the 5- and 8-positions, the 6- and 7-positions, the 6- and 8-positions, or the 7- and 8-positions; a difluoro-1-naphthyl group having two fluorine atoms at the 2- and 3-positions, the 2- and 4-positions, the 2- and 5-positions, the 2- and 6-positions, the 2- and 7-positions, the 2- and 8-positions, the 3- and 4-positions, the 3- and 5-positions, the 3- and 6-positions, the 3- and 7-positions, the 3- and 8-positions, the 4- and 5-positions, the 4- and 6-positions, the 4- and 7-positions, the 4- and 8-positions, the 5- and 6-positions, the 5- and 7-positions, the 5- and 8-positions, the 6- and 7-positions, the 6- and 8-positions, or the 7- and 8-positions; a tri(trifluoromethyl)-1-naphthyl group having three trifluoromethyl groups at the 2-, 3- and 4-positions, the 2-, 3- and 5-positions, the 2-, 3- and 6-positions, the 2-, 3- and 7-positions, the 2-, 3- and 8-positions, the 2-, 4- and 5-positions, the 2-, 4- and 6-positions, the 2-, 4- and 7-positions, the 2-, 4- and 8-positions, the 2-, 5- and 6-positions, the 2-, 5- and 7-positions, the 2-, 5- and 8-positions, the 2-, 6- and 7-positions, the 2-, 6- and 8-positions, the 3-, 4- and 5-positions, the 3-, 4- and 6-positions, the 3-, 4- and 7-positions, the 3-, 4- and 8-positions, the 3-, 5- and 6-positions, the 3-, 5- and 7-positions, the 3-, 5- and 8-positions, the 3-, 6- and 7-positions, the 3-, 6- and 8-positions, the 3-, 7- and 8-positions, the 4-, 5- and 6-positions, the 4-, 5- and 7-positions, the 4-, 5- and 8-positions, the 4-, 6- and 7-positions, the 4-, 6- and 8-positions, the 4-, 7- and 8-positions, the 5-, 6- and 7-positions, the 5-, 6- and 8-positions, the 5-, 7- and 8-positions, and the 6-, 7- and 8-positions; a trifluoro-1-naphthyl group having three fluorine atoms at the 2-, 3- and 4-positions, the 2-, 3- and 5-positions, the 2-, 3- and 6-positions, the 2-, 3- and 7-positions, the 2-, 3- and 8-positions, the 2-, 4- and 5-positions, the 2-, 4- and 6-positions, the 2-, 4- and 7-positions, the 2-, 4- and 8-positions, the 2-, 5- and 6-positions, the 2-, 5- and 7-positions, the 2-, 5- and 8-positions, the 2-, 6- and 7-positions, the 2-, 6- and 8-positions, the 3-, 4- and 5-positions, the 3-, 4- and 6-positions, the 3-, 4- and 7-positions, the 3-, 4- and 8-positions, the 3-, 5- and 6-positions, the 3-, 5- and 7-positions, the 3-, 5- and 8-positions, the 3-, 6- and 7-positions, the 3-, 6- and 8-positions, the 3-, 7- and 8-positions, the 4-, 5- and 6-positions, the 4-, 5- and 7-positions, the 4-, 5- and 8-positions, the 4-, 6- and 7-positions, the 4-, 6- and 8-positions, the 4-, 7- and 8-positions, the 5-, 6- and 7-positions, the 5-, 6- and 8-positions, the 5-, 7- and 8-positions, and the 6-, 7- and 8-positions; and 2, 3, 4, 5, 6, 7, 8-heptafluoro-1-naphthyl group.

Among the naphthyl groups represented by general formula (5), 2-naphthyl groups include, for example: a (trifluoromethyl)-2-naphthyl group having a trifluoromethyl group at the 1-, 3-, 4-, 5-, 6-, 7- or 8-position; a fluoro-2-naphthyl group having a fluorine atom at the 1-, 3-, 4-, 5-, 6-, 7-or 8-position; a bis(trifluoromethyl)-2-naphthyl group having two trifluoromethyl groups at the 1- and 3-positions, the 1- and 4-positions, the 1- and 5-positions, the 1- and 6-positions, the 1- and 7-positions, the 1- and 8-positions, the 3- and 4-positions, the 3- and 5-positions, the 3- and 6-positions, the 3- and 7-positions, the 3- and 8-positions, the 4- and 5-positions, the 4- and 6-positions, the 4- and 7-positions, the 4- and 8-positions, the 5- and 6-positions, the 5- and 7-positions, the 5- and 8-positions, the 6- and 7-positions, the 6- and 8-positions, or the 7- and 8-positions; a difluoro-2-naphthyl group having two fluorine atoms at the 1- and 3-positions, the 1- and 4-positions, the 1- and 5-positions, the 1- and 6-positions, the 1- and 7-positions, the 1- and 8-positions, the 3- and 4-positions, the 3- and 5-positions, the 3- and 6-positions, the 3- and 7-positions, the 3- and 8-positions, the 4- and 5-positions, the 4- and 6-positions, the 4- and 7-positions, the 4- and 8-positions, the 5- and 6-positions, the 5- and 7-positions, the 5- and 8-positions, the 6- and 7-positions, the 6- and 8-positions, or the 7- and 8-positions; a tri(trifluoromethyl)-2-naphthyl group having three trifluoromethyl groups at the 1-, 3- and 4-positions, the 1-, 3- and 5-positions, the 1-, 3- and 6-positions, the 1-, 3- and 7-positions, the 1-, 3- and 8-positions, the 1-, 4- and 5-positions, the 1-, 4- and 6-positions, the 1-, 4- and 7-positions, the 1-, 4- and 8-positions, the 1-, 5- and 6-positions, the 1-, 5- and 7-positions, the 1-, 5- and 8-positions, the 1-, 6- and 7-positions, the 1-, 6- and 8-positions, the 3-, 4- and 5-positions, the 3-, 4- and 6-positions, the 3-, 4- and 7-positions, the 3-, 4- and 8-positions, the 3-, 5- and 6-positions, the 3-, 5- and 7-positions, the 3-, 5- and 8-positions, the 3-, 6- and 7-positions, the 3-, 6- and 8-positions, the 3-, 7- and 8-positions, the 4-, 5- and 6-positions, the 4-, 5- and 7-positions, the 4-, 5- and 8-positions, the 4-, 6- and 7-positions, the 4-, 6- and 8-positions, the 4-, 7- and 8-positions, the 5-, 6- and 7-positions, the 5-, 6- and 8-positions, the 5-, 7- and 8-positions, and the 6-, 7- and 8-positions; a trifluoro-2-naphthyl group having three fluorine atoms at the 1-, 3- and 4-positions, the 1-, 3- and 5-positions, the 1-, 3- and 6-positions, the 1-, 3- and 7-positions, the 1-, 3- and 8-positions, the 1-, 4- and 5-positions, the 1-, 4- and 6-positions, the 1-, 4- and 7-positions, the 1-, 4- and 8-positions, the 1-, 5- and 6-positions, the 1-, 5- and 7-positions, the 1-, 5- and 8-positions, the 1-, 6- and 7-positions, the 1-, 6- and 8-positions, the 3-, 4- and 5-positions, the 3-, 4- and 6-positions, the 3-, 4- and 7-positions, the 3-, 4- and 8-positions, the 3-, 5- and 6-positions, the 3-, 5- and 7-positions, the 3-, 5- and 8-positions, the 3-, 6- and 7-positions, the 3-, 6- and 8-positions, the 3-, 7- and 8-positions, the 4-, 5- and 6-positions, the 4-, 5- and 7-positions, the 4-, 5- and 8-positions, the 4-, 6- and 7-positions, the 4-, 6- and 8-positions, the 4-, 7- and 8-positions, the 5-, 6- and 7-positions, the 5-, 6- and 8-positions, the 5-, 7- and 8-positions, and the 6-, 7- and 8-positions; and 1, 3, 4, 5, 6, 7, 8-heptafluoro-2-naphthyl group.

In the Nile Red luminescent compound emitting red light, —NR¹R² is an electron donating group and —Ar is an electron attractive group. Therefore, π electron cloud on the skeleton of the Nile Red is extended over the substituents, which results in a super resonance effect. Thus, the application of a little energy enables the luminescent compound to emit red light. The novel luminescent compound of this invention is characterized by a structure where R¹—N—R², the electron donating group, provides the π electron cloud with electrons and Ar, the aromatic electron attractive compound, attracts electrons from the π electron cloud. Because this Nile Red skeleton has an electronically stable structure and therefore the Nile Red luminescent compound is chemically stable, the luminescent compound does not deteriorate even under severe environments, which is a special character of the compound.

The Nile Red luminescent compound emitting red light represented by general formula (1) may be prepared by the following method.

The compound may be obtained by reacting a Nile Red compound represented by general formula (6) with the electron attractive aromatic acetonitrile represented by general formula (7).

The Nile Red compound and the electron attractive aromatic acetonitrile react easily by heating them in a solvent. The solvent includes acetic anhydride, acetic acid, an acid anhydride having not more than 5 carbon atoms, an aromatic solvent such as benzene or toluene, adioxane, etc. The reaction temperature usually ranges between 100 and 250° C., preferably between 100 and 170° C. After the reaction, the purification and separation by an ordinary method will provide the aimed Nile Red luminescent compound.

The Nile Red luminescent compound in accordance with this invention can easily be produced only by heating a mixture of the Nile-red compound and the electron attractive aromatic acetonitrile. A simple production method such as the above-mentioned is an industrial method.

The Nile Red luminescent compound red light in accordance with this invention is used for a luminescent element. This luminescent element comprises a cathode, a luminescent layer including the luminescent compound and an anode formed on the luminescent layer. This luminescent element may employ various structures, as long as it has the luminescent layer including the Nile Red luminescent compound. One example of the luminescent element is a one-layer organic luminescent element having, between the cathode and the anode, a luminescent layer including an electron-transporting substance that transports electrons, the Nile Red luminescent compound, and a hole-transporting substance that transports holes. Another example is a two-layer organic luminescent element having, between the cathode and the anode, a hole-transporting layer including a hole-transporting substance, and an electron-transporting luminescent layer including the Nile Red luminescent compound, the electron-transporting luminescent layer being laminated on the hole-transporting layer, such as a two-layer pigment-doped luminescent element having, between the cathode and the anode, a hole-transporting layer including a hole-transporting substance, and a luminescent layer that includes a host pigment and the Nile Red luminescent compound as a guest pigment, the luminescent layer being laminated on the hole-transporting layer. A further example is a two-layer organic luminescent element having, between the cathode and the anode, an electron-transporting layer including an electron-transporting substance, and a hole-transporting luminescent layer including the Nile Red luminescent compound, the electron-transporting luminescent layer being laminated on the hole-transporting layer, such as a two-layer pigment-doped luminescent element having, between the cathode and the anode, an electron-transporting layer including an electron-transporting substance, and a luminescent layer that includes a host pigment and the Nile Red luminescent compound as a guest pigment, the luminescent layer being laminated on the hole-transporting layer. A still further example is a three-layer luminescent element having, between the cathode and the anode, a hole-transporting layer, a luminescent layer including the Nile Red luminescent compound, and an electron-transporting layer. In the examples above, a laminate comprising one luminescent layer, two layers and three layers is sometimes called an organic layer.

Typically, this luminescent element may be formed on a substrate. The substrate includes a transparent one made of glass, plastic, etc.

For the cathode, various materials may be employed, as long as their work functions are large, they are transparent, and they can dope holes to the laminate by the application of voltage. Specifically, the cathode may be made of a transparent inorganic conductive material of ITO, In₂O₃, SnO₂, ZnO, CdO, etc. and derivatives thereof, or an electrically conductive high polymer such as polyaniline.

The thickness of the cathode film is typically 100-200 μm, the surface resistance is 10-20 Ω/□.

This cathode may be formed on the substrate by chemical vapor phase deposition, spray pyrolysis, high-vacuum metal deposition, electron beam deposition, sputtering, ion beam sputtering, ion plating, ion-assisted deposition, and other methods.

For the anode may be employed a material having a small work function. Examples of the material are elementary metals and metallic alloys, such as MgAg, aluminum alloy, metallic calcium, etc. Preferable anode is an alloy of aluminum and a small amount of lithium. This anode may easily be formed on the surface of the organic layer including the luminescent layer laminated on the substrate, by the technique of metal deposition.

The electron-transporting substance includes, for example, an oxadiazole derivative such as 2,5-bis (1-naphthyl)-1,3,4-oxadiazole (BND) and 2-(4-tert-butylphenyl)-5-(4-biphenylyl)-1,3,4-oxadiazole, and 2,5-bis(5′-tert-butyl-2′-benzoxazolyl)thiophene. Also, a metal complex material such as quinolinol aluminum complex (Alq3), benzoquinolinol beryllium complex (Bebq2) can be used suitably.

The hole-transporting substance includes a triphenylamine compound such as N,N′-diphenyl-N,N′-di(m-tolyl)-benzidine (TPD) and α-NPD, a hydrazon compound, a stilbene compound, a heterocyclic compound, a π electron star burst positive hole transporting substance, etc.

The organic layer in the luminescent element may be formed through deposition or application such as a spin cast method, a coating method and dipping method.

When either of the deposition or the application is employed, a buffer layer should be inserted between the electrodes and the organic layer.

Materials for the buffer layer between the anode and the organic layer are, for example, an alkaline metal compound such as lithium fluoride, an alkaline earth metal compound such as magnesium fluoride, an oxide such as an aluminum oxide, and 4,4′-biscarbazole biphenyl(Cz-TPD). Also, materials for forming the buffer layer between the cathode made of ITO, etc. and the organic layer are, for example, derivatives of m-MTDATA (4,4′,4″-tris(3-methylphenylphenylamino)triphenyl amine), phthalocyanine, polyaniline, and polythiophene, and inorganic oxides such as molybdenum oxide, ruthenium oxide, vanadium oxide and lithium fluoride. When the materials are suitably selected, these buffer layers can lower the driving voltage of the luminescent element, improve the quantum efficiency of luminescence, and achieve an increase in the luminance of the emitted light.

A preferable luminescent element that can be formed by deposition comprises a cathode made of ITO, etc. by vapor deposition, a hole-transporting layer formed on the surface of the cathode, preferably with a buffer layer sandwiched between the cathode and the hole-transporting layer, an electron-transporting luminescent layer formed on the surface of the hole-transporting layer by deposition, which electron-transporting luminescent layer includes the host pigment such as Alq3 or Bebq2 and the Nile Red luminescent compound, the guest pigment, and an anode formed on the surface of the electron-transporting luminescent layer by vapor deposition, preferably with a buffer layer sandwiched between the cathode and the electron-transporting luminescent layer.

The amount of the Nile Red luminescent compound included in the electron-transporting luminescent layer of this element is usually 0.001-50 weight %, preferably 0.01-10 weight %, based on the weight of the host pigment. When the amount of the Nile Red luminescent compound is within the range, the compound emits especially clear red light.

The thickness of the electron-transporting luminescent layer is usually 30-300 nm, preferably 50-200 nm. When the thickness is within the range, the application of a low voltage can provide emission at a high luminance.

A preferable element that can be formed by application comprises a cathode, such as that made of ITO, formed on the surface of the substrate; an organic layer formed on the surface of the cathode by coating the surface with a solution containing the Nile Red luminescent compound, the electron-transporting material and the hole-transporting high polymer, and drying the coated surface; and an anode laminated, preferably on a buffer layer formed on the organic layer, by vapor deposition.

The amount of the Nile Red luminescent compound included in the organic layer is usually 0.01-10 weight %, preferably 0.1-5 weight %. When the amount of the Nile Red luminescent compound is within the range, the compound emits especially clear red light.

Examples of the hole-transporting high polymer are polyvinyl carbazole and poly(3-alkylenethiophene). Also, it is preferable, if the organic layer includes rubrene, especially both of rubrene and Alq3.

The thickness of this electron-transporting luminescent layer is usually 30-300 nm, preferably 50-200 nm. When the thickness is within the range, the application of a low voltage can provide emission at a high luminance.

The luminescent element in accordance with this invention can generally be used for an element driven by a direct current. It can also be used as an element driven by a pulse and one driven by an alternate current. Conventional Nile Red elements only emitted orange light. The luminescent element of this invention, since it includes the Nile Red luminescent compound, emits a red close to crimson at a high luminance.

When the luminescent layer of the luminescent element in accordance with this invention only includes the Nile Red luminescent compound, the luminescent layer emits red light. When this luminescent layer includes the Nile Red luminescent compound, a blue light-emitting compound and a green light-emitting compound, the layer emits white light.

The blue light-emitting compound includes a diphenylvinyl biphenol compound emitting blue light and a stilbene compound emitting blue light. An example of preferable diphenylvinyl biphenol compounds emitting blue light is DPVBi represented by formula (10).

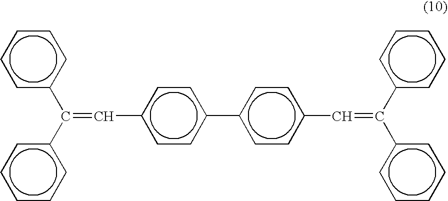

The green light-emitting compound includes a coumarin compound emitting green light, an indophenol compound emitting green light and an indigo compound emitting green light. A preferable one is a coumarin compound emitting green light represented by formula (11).

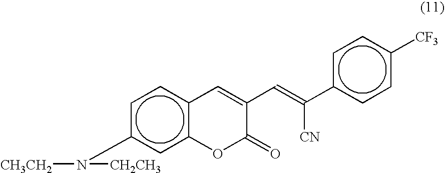

To make the luminescent element of this invention emit white light, the weight ratio of the Nile Red luminescent compound to the blue light-emitting compound to the green light-emitting compound in the luminescent layer should usually be 5-200:10-100:50-20000, preferably 10-100: 50-500:100-10000.

The element emitting red light and the one emitting white light are used for illuminators, displays, etc.

### EXAMPLES

### Working Example 1

Synthesis of Nile Red Luminescent Compound A

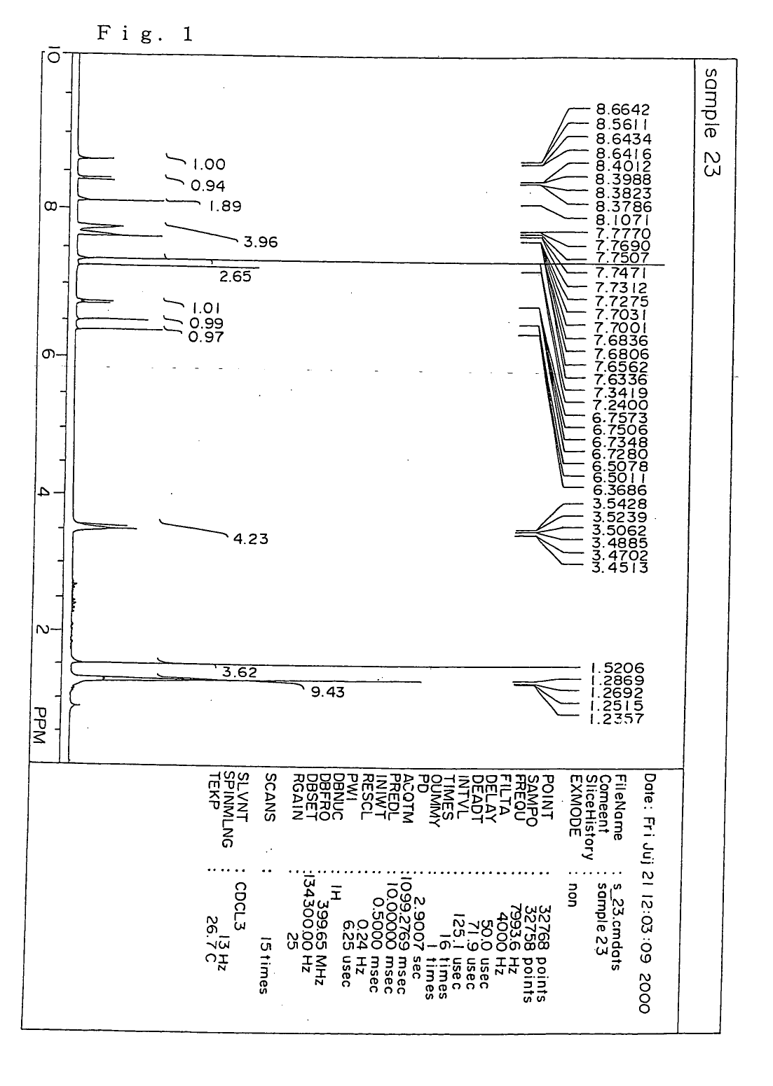

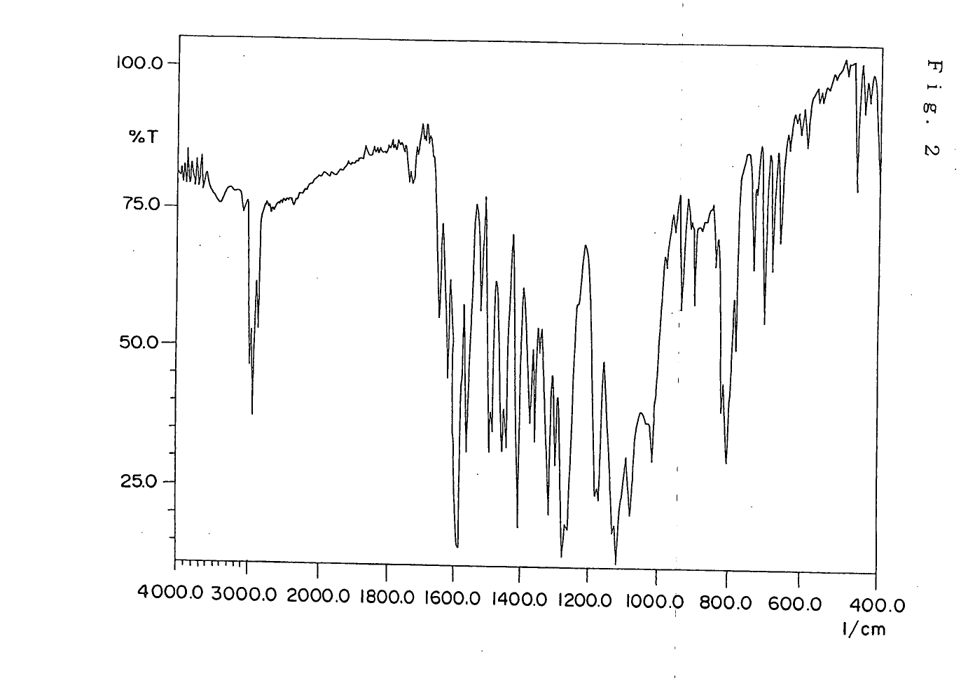

0.50 g (1.57 mmol) of Nile Red, 0.40 g (1.57 mmol) of 3,5-bis(trifluoromethyl)phenylacetonitrile, and 25 ml of acetic anhydride were placed in a 100 ml pear-shaped flask. The solution in the pear-shaped flask was heated in a silicone oil bath to 135° C. and allowed to react for 4 hours. Acetic an hydride was distilled away with an evaporator and the remaining was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 30 mg of violaceous solid was obtained in a 12% yield. The melting point of the product was 257-260° C. A ¹H-NMR spectrum and an IR spectrum of this product are shown in FIGS. 1 and 2. The results of elemental analysis of this product are as follows. Based on these results, the obtained product was identified as the chemical compound represented by formula (12).

The results of elemental analysis

Found values: C: 66.03, H: 4.31, N: 5.16

Calculated values: C: 65.91, H: 4.20, N: 5.30

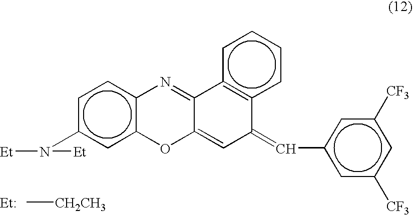

### Working Example 2

Synthesis of Nile Red Luminescent Compound B

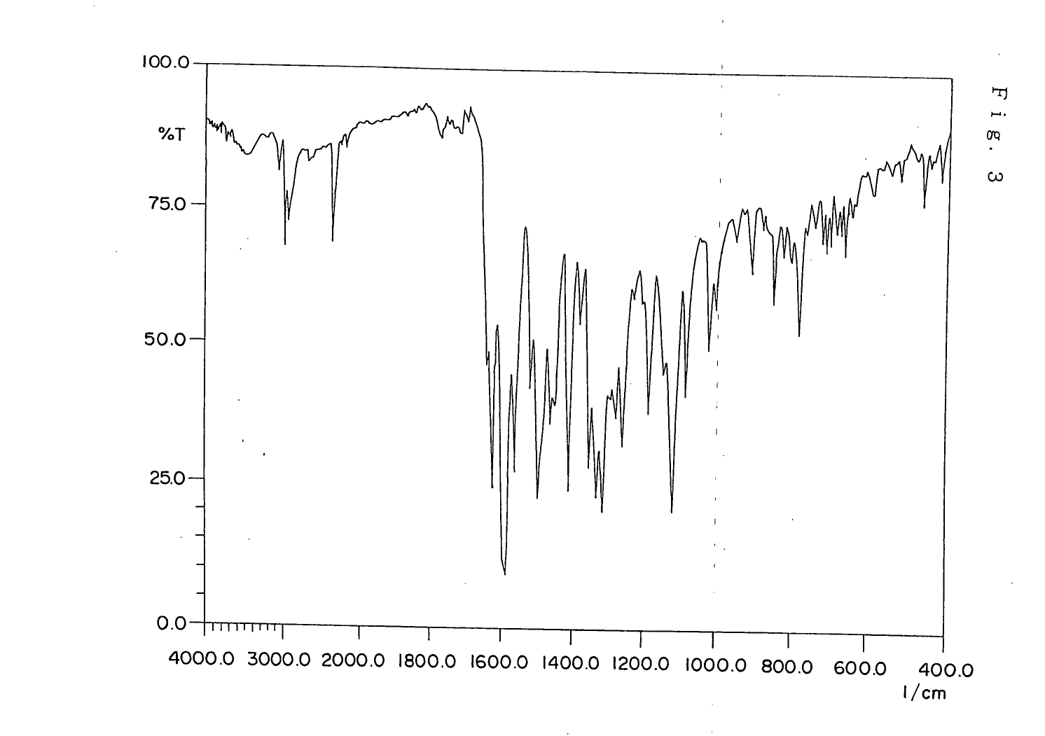

0.50 g (1.57 mmol) of Nile Red, 0.35 g (1.57 mmol) of 2,3-difluoro-4-(trifluoromethyl)phenylacetonitrile, and 25 ml of acetic anhydride were placed in a 100 ml pear-shaped flask. The solution in the pear-shaped flask was heated in a silicone oil bath to 135° C. and allowed to react for 3 hours. Acetic an hydride was distilled away with an evaporator and the remaining was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 10 mg of violaceous solid was obtained in a 6.4% yield. The melting point of the product was 172-174° C. An IR spectrum of this product is shown in FIG. 3. The results of elemental analysis of this product are as follows. Based on these results, the obtained product was identified as the chemical compound represented by formula (13).

The results of elemental analysis

Found values: C: 67.90, H: 4.23, N: 5.65

Calculated values: C: 67.74, H: 4.26, N: 5.64

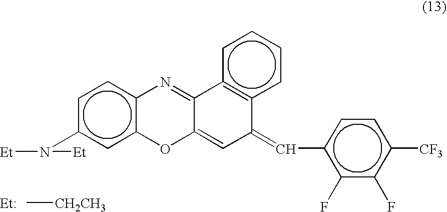

### Working Example 3

In a 5 ml graduated flask were placed 70 mg of polyvinyl carbazole, a product produced by Kanto Kagaku Co., Ltd., which polyvinyl carbazole will be abbreviated to PVK, 29 mg of 2,5-bis(1-naphthyl)-1,3,4-oxadiazole synthesized by the inventors, which will be abbreviated to BND, and 1 mg of Nile Red luminescent compound A. Dichloroethane was added to the mixture so that the total volume of the mixture was 5 ml. Thus, a solution containing the Nile Red luminescent compound was prepared. This solution was sufficiently homogenized by irradiating it with ultrasound for 20 minutes using a model US-2 ultrasonic cleaner, a product by SND Co. An ITO substrate, a product by Sanyo Shinku Industries, Co., Ltd., of which dimensions were 50×50 mm, was ultrasonically cleaned in acetone for 10 minutes and then in 2-propanol for 10 minutes. The substrate was blow-dried with nitrogen. Then, the substrate was cleaned by ultraviolet-light irradiation for 30 seconds at a wavelength of 172 nm with a UV irradiator produced by M.D. Excimer Inc. The solution containing the Nile Red luminescent compound was dropped onto the obtained ITO substrate and a film was formed on the surface of the substrate by spin-coating at 1,500 rpm for 3 seconds with a model 1H-D7 spin coater, a product by Mikasa Co., Ltd. The substrate with the film was dried for 30 minutes in a constant temperature bath of 50° C. The electrode of an aluminum alloy, a product by Kojundo Chemical Laboratory, Co., Ltd., in which the weight ratio of Al to Li was 99:1, was vapor-deposited on the substrate under 4×10-6-Torr with a vacuum metallizer (Model VDS-M2-46, produced by DIAVAC Limited). The thickness of the electrode was 1,500 Å. Thus an EL element was obtained.

The luminance and the chromaticity of the EL element were measured with a Fast BM-7 measuring apparatus produced by TOPCON Corporation, with the voltage being raised gradually. The results are shown in Table 2.

### Working Example 4

In a 5 ml graduated flask were placed 68 mg of PVK, 31.2 mg of 2-(4-tert-butylphenyl-5-(4-biphenylyl)-1,3,4-oxadiazole, which will be abbreviated to PBD, and 0.8 mg of Nile Red luminescent compound A. Dichloroethane was added to the mixture so that the total volume of the mixture was 5 ml. An EL element was prepared from the obtained solution containing the Nile Red luminescent compound, and the luminance and the chromaticity thereof were measured in the same way as that explained in Example 3 above. The results are shown in Table 2.

### Working Example 5

In a 5 ml graduated flask were placed 63.7 mg of PVK, 35.5 mg of 2,5-bis (5′-tert-butyl-2′-benzoxazolyl)thiophene, which will be abbreviated to BBOT, and 0.8 mg of Nile Red luminescent compound A. Dichloroethane was added to the mixture so that the total volume of the mixture was 5 ml. An EL element was prepared from the obtained solution containing the Nile Red luminescent compound, and the luminance and the chromaticity thereof were measured in the same way as that explained in Example 3 above. The results are shown in Table 2.

### Working Example 6

In a 5 ml graduated flask were placed 64.0 mg of PVK, 35.6 mg of BBOT, and 0.4 mg of Nile Red luminescent compound A. Dichloroethane was added to the mixture so that the total volume of the mixture was 5 ml. An EL element was prepared from the obtained solution containing the Nile Red luminescent compound, and the luminance and the chromaticity thereof were measured in the same way as that explained in Example 3 above. The results are shown in Table 2.

### Comparative Example 1

In a 5 ml graduated flask were placed 68.2 mg of PVK, 31.3 mg of PBD, and 0.5 mg of Nile Red. Dichloroethane was added to the mixture so that the total volume of the mixture was 5 ml. An EL element was prepared from the obtained solution containing Nile Red, and the luminance and the chromaticity thereof were measured in the same way as that explained in Example 3 above. The results are shown in Table 2.

TABLE 2

| Amount mg | W. Ex. 3 | W. Ex. 4 | W. Ex. 5 | W. Ex. 6 | C. Ex. 1 |
|---|---|---|---|---|---|
| PVK | 70.0 | 68.0 | 63.7 | 64.0 | 68.2 |
| BND | 29.0 | | | | |
| PBD | | 31.2 | | 31.3 | |
| BBOT | | | 35.5 | 35.6 | |
| Pigment A | 1.0 | 0.8 | 0.8 | 0.4 | |
| Nile-red | | | | | 0.5 |
| compound | | | | | |
| The highest | 2454.4 | 2215.0 | 2629.2 | 3475.6 | 2137.0 |
| luminance | | | | | |
| cd/m² | | | | | |
| Chromaticity | | | | | |
| X | 0.6417 | 0.6307 | 0.6330 | 0.6278 | 0.5402 |
| Y | 0.3477 | 0.3535 | 0.3610 | 0.3648 | 0.4324 |

### Working Example 7

In a 5 ml graduated flask were placed 70.1 mg of polyvinyl carbazole, a product produced by Kanto Kagaku Co., Ltd., which polyvinyl carbazole will be abbreviated to PVK, 29.3 mg of 2,5-bis(1-naphthyl)-1,3,4-oxadiazole synthesized by the inventors, which will be abbreviated to BND, and 0.61 mg of Nile Red luminescent compound A. Dichloroethane was added to the mixture so that the total volume of the mixture was 5 ml. Thus, a solution containing the Nile Red luminescent compound was prepared. This solution was sufficiently homogenized by irradiating it with ultrasound for 20 minutes using a model US-2 ultrasonic cleaner, a product by SND Co. An ITO substrate, a product by Sanyo Shinku Industries, Co., Ltd., of which dimensions were 50×50 mm, was ultrasonically cleaned in acetone for 10 minutes and then in 2-propanol for 10 minutes. The substrate was blow-dried with nitrogen. Then, the substrate was cleaned by ultraviolet-light irradiation for 30 seconds at a wavelength of 172 nm with a UV irradiator produced by M.D. Excimer Inc. The solution containing the Nile Red luminescent compound was dropped onto the obtained ITO substrate and a film was formed on the surface of the substrate by spin-coating at 1,500 rpm for 3 seconds with a model 1H-D7 spin coater, a product by Mikasa Co., Ltd. The substrate with the film was dried for 30 minutes in a constant temperature bath of 50° C. The electrode of an aluminum alloy, a product by Kojundo Chemical Laboratory, Co., Ltd., in which the weight ratio of Al to Li was 99:1, was vapor-deposited on the substrate under 4×10-6 torr with a vacuum metallizer (Model VDS-M2-46, produced by DIAVAC Limited). The thickness of the electrode was 1,500 Å. Thus an EL element was obtained.

The luminance and the chromaticity of the EL element were measured with a Fast BM-7 measuring apparatus produced by TOPCON Corporation, with the voltage being raised gradually. The results are shown in Table 3.

### Working Example 8

In a 5 ml graduated flask were placed 69.9 mg of PVK, 29.1 mg of BND, 0.4 mg of rubrene and 0.6 mg of Nile Red luminescent compound A. Dichloroethane was added to the mixture so that the total volume of the mixture was 5 ml. An EL element was prepared from the obtained solution containing the Nile Red luminescent compound, and the luminance and the chromaticity thereof were measured in the same way as that explained in Example 7 above. The results are shown in Table 3.

### Working Example 9

The steps of Working Example 7 were repeated, except that Nile Red luminescent compound B was used instead of Nile Red luminescent compound A. The results of the measurements are shown in Table 3.

### Working Example 10

Synthesis of Nile Red Luminescent Compound C

The steps of Working Example 1 were repeated, except that 1.57 mmol of 2,4-bis(trifluoromethyl)phenylacetonitrile was used instead of 3,5-bis (trifluoromethyl)phenylacetonitrile. Thus, Nile Red luminescent compound C represented by formula (14) was synthesized.

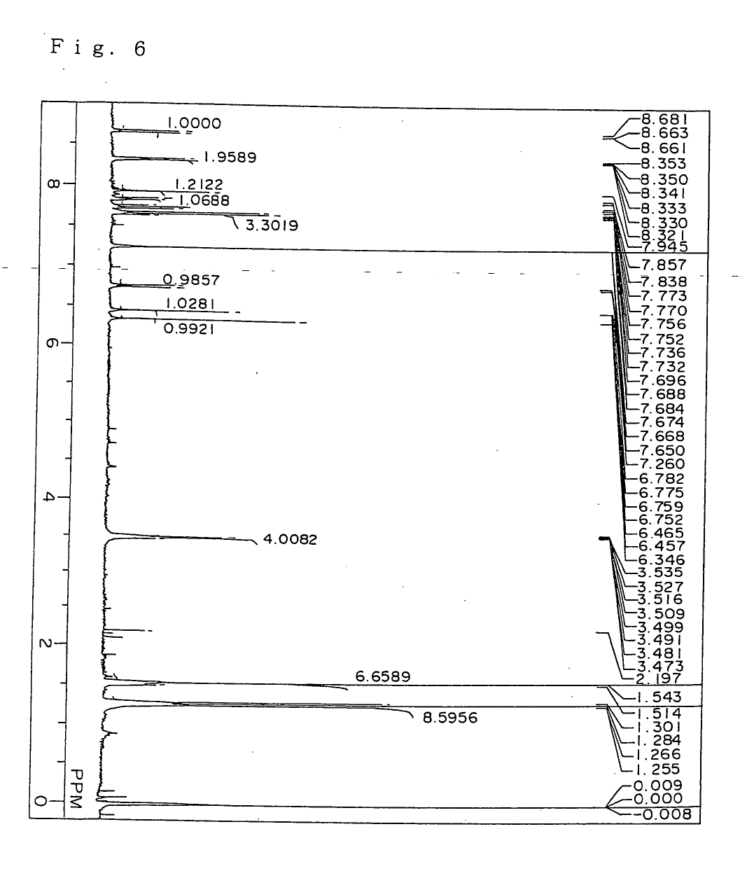

A ¹H-NMR spectrum of this product is shown in FIG. 6. The results of elemental analysis of this product are as follows.

Found values: C: 66.13, H: 4.21, N: 5.18

Calculated values: C: 65.91, H: 4.20, N: 5.30

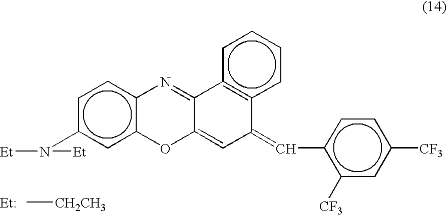

The steps of Working Example 7 were repeated, except that Nile Red luminescent compound C was used instead of Nile Red luminescent compound A. Thus, an EL element was obtained. The luminance and the chromaticity of this EL element were measured in the same way. The results of the measurements are shown in Table 3.

TABLE 3

| Amount mg | W. Ex. 7 | W. Ex. 8 | W. Ex. 9 | W. Ex. 10 |
|---|---|---|---|---|
| PVK | 70.1 | 69.9 | 70.1 | 70.1 |
| BND | 29.3 | 29.1 | 29.3 | 29.3 |
| Rubrene | | 0.4 | | |
| Luminescent | 0.6 | 0.6 | | |
| compound A | | | | |
| Luminescent | | | 0.6 | |
| compound B | | | | |
| Luminescent | | | | 0.6 |
| compound C | | | | |
| The highest | 3006.0 | 2015.0 | 1349.0 | 2985.0 |
| luminance | | | | |
| cd/m² | | | | |
| Chroruaticity | | | | |
| X | 0.6248 | 0.6694 | 0.6294 | 0.6218 |
| Y | 0.3453 | 0.3152 | 0.3458 | 0.3636 |

### Working Example 11

An ITO substrate, which had been cleaned in the same way as that of Working Example 7, was set in a vacuum metallizer, and N,N′-diphenyl-N,N-di(m-tolyl)-benzidine (TPD) was deposited on the substrate under not more than 1×₁₀-6 torr. The thickness of the deposited film was 60 nm. Then, a mixture of tris (8-quinolinate) aluminum (Alq3) and Nile Red luminescent compound A, wherein the amount of the latter was 1.7 weight %, was further deposited on the surface of the TPD film, so that the thickness of the film of the mixture was 31 nm. Finally, aluminum electrode was deposited in a thickness of 150 nm. Thus, an EL element was prepared.

The highest luminance and the chromaticity of this EL element were measured in the same way as that in Working Example 7. The results are shown in Table 4.

### Working Example 12

An EL element was prepared in the same way as that of Working Example 11, except that the amount of Nile Red luminescent compound A in the mixture of Alq3 and compound A was 1.5 weight %, and the mixture was deposited on the substrate so that the thickness of the film was 23 nm. The results are shown in Table 4.

TABLE 4

| | | W. Ex. 11 | W. Ex. 12 |
|---|---|---|---|
| Thickness of | TPF | 60 | 70 |
| film (nm) | Alq3 + Luminescent Cpd. A | 31 | 23 |
| Concentrations | Alq3 | 98.5 | 99.4 |
| in the mixture | Luminescent compound A | 1.5 | 0.6 |
| The highest luminance (cd/m²) | | 623.0 | 4427.0 |
| Chromaticity | X | 0.6536 | 0.6155 |
| | Y | 0.3184 | 0.3459 |

### Working Example 13

In a 5 ml graduated flask were placed 70.0 mg of poly(N-vinyl carbazole), a product produced by Kanto Kagaku Co., Ltd., which poly(N-vinyl carbazole) will be abbreviated to PVK, 14.85 mg of 2,5-bis(1-naphthyl)-1,3,4-oxadiazole, a product produced by Lancaster, which will be abbreviated to BND, 0.05 mg of Nile Red luminescent compound A, 0.10 mg of a green light-emitting pigment represented by formula (9), which will be called “pigment B”, and 15.0 mg of a blue light-emitting pigment represented by formula (8), which will be abbreviated to DPVBi. Dichloroethane was added to the mixture so that the total volume of the mixture was 5 ml. This solution was sufficiently homogenized by irradiating it with ultrasound for 20 minutes using a model US-2 ultrasonic cleaner, a product by SND Co.

An ITO substrate was ultrasonically cleaned in acetone for 10 minutes and then in IPA for 10 minutes. The substrate was blow-dried with nitrogen. Then, the substrate was cleaned by ultraviolet-light irradiation for 5 minutes with a model PL16-110 photo surface processor produced by SEN LIGHTS CORPORATION. The solution was dropped onto the obtained ITO substrate and a film was formed on the surface of the substrate by spin-coating with a model 1H-D7 spin coater, a product by Mikasa Co., Ltd. The substrate with the film was dried for 30 minutes in a constant temperature bath of 50° C. The electrode of an aluminum alloy, a product by Kojundo Chemical Laboratory, Co., Ltd., in which the weight ratio of Al to Li was 99:1, was vapor-deposited on the substrate under a pressure of not more than 10-6 Torr with a vacuum metallizer (Model VDS-M2-46, produced by DIAVAC Limited). The thickness of the electrode was 150 nm. Thus an EL element was obtained.

The optical properties of the EL element were measured with a Fast BM-7 measuring apparatus produced by TOPCON Corporation. The results are shown in Table 5. As understood from Table 5, the combination of the Nile Red luminescent compound in accordance with this invention, the green light-emitting pigment and the blue light-emitting pigment could provide an EL element capable of emitting white light.

### Working Example 14

In a 5 ml graduated flask were placed 70.1 mg of PVK, 14.85 mg of BND, 0.04 mg of Nile Red luminescent compound A, 0.10 mg of the green light-emitting pigment, which was the same as that used in Working Example 13, and 15.0 mg of the blue light-emitting pigment, which was the same as that used in Working Example 13. Dichloroethane was added to the mixture so that the total volume of the mixture was 5 ml. An EL element was prepared in the same manner as that of Working Example 13 and the optical properties thereof were measured. The results are shown in Table 5. As understood from Table 4, the combination of the Nile Red luminescent compound in accordance with this invention, the green light-emitting pigment and the blue light-emitting pigment could provide an EL element capable of emitting white light.

### Working Example 15

In a 5 ml graduated flask were placed 70.0 mg of PVK, 20.0 mg of BND, 0.02 mg of Nile Red luminescent compound A, 0.03 mg of the green light-emitting pigment, which was the same as that used in Working Example 13, and 9.95 mg of the blue light-emitting pigment, which was the same as that used in Working Example 13. Dichloroethane was added to the mixture so that the total volume of the mixture was 5 ml. An EL element was prepared in the same manner as that of Working Example 13 and the optical properties thereof were measured. The results are shown in Table 5. As understood from Table 4, the combination of the Nile Red luminescent compound in accordance with this invention, the green light-emitting pigment and the blue light-emitting pigment could provide an EL element capable of emitting white light.

TABLE 5

| | | W. Ex. 13 | W. Ex. 14 | W. Ex. 15 |
|---|---|---|---|---|
| PVK | | 70.0 | 70.1 | 70.0 |
| BND | | 14.85 | 14.85 | 20.0 |
| Pigment A | | 0.05 | 0.04 | 0.02 |
| Pigment B | | 0.10 | 0.10 | 0.03 |
| DPVBi | | 15.0 | 15.0 | 9.95 |
| The highest | | 4867.0 | 3631.0 | 2819.0 |
| luminance (cd/m²) | | | | |
| Chromaticity | x | 0.3788 | 0.3616 | 0.3101 |
| | y | 0.3547 | 0.3399 | 0.3035 |

### Working Example 16

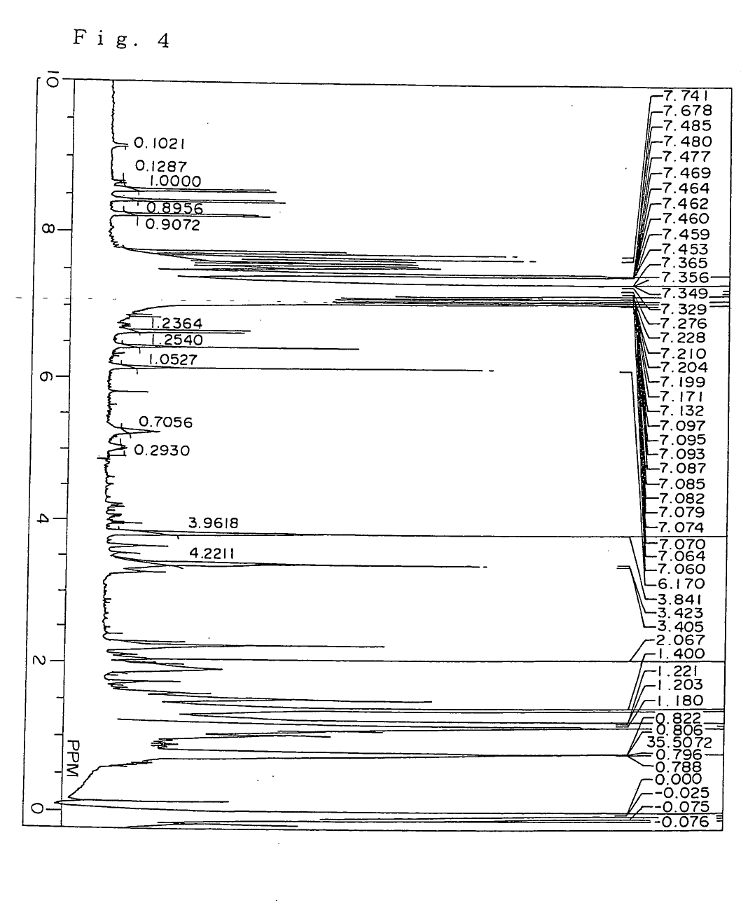

0.92 g (2.89 mmol) of Nile Red, 11.0 g (4.33 mmol) of fluorinated phenylacetonitrile (1) represented by formula (15), and 50 ml of acetic anhydride were placed in a 100 ml pear-shaped flask. The solution in the pear-shaped flask was heated in a silicone oil bath to 135° C. and allowed to react for 1 hour. Acetic anhydride was distilled away with an evaporator and the residue was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 40 mg of violaceous solid was obtained. The melting point of the product was 235-240° C. An NMR chart of this product is shown in FIG. 4. The results of elemental analysis of this violaceous product areas follows. Based on these results, the obtained product was identified as the Nile Red luminescent compound represented by formula (16).

The results of elemental analysis

Found values: C: 68.90, H: 4.28, N: 5.49

Calculated values: C: 68.77, H: 4.38, N: 5.53

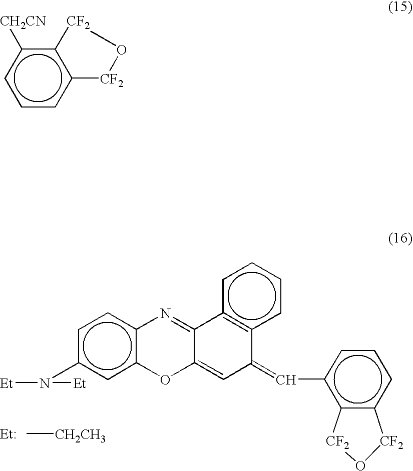

### Working Example 17

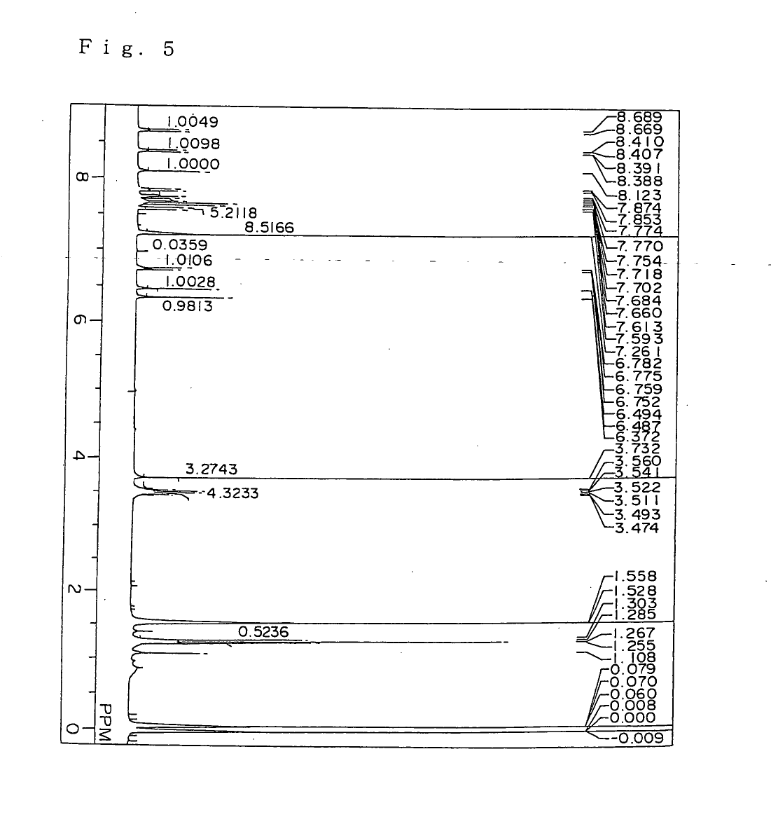

0.92 g (2.89 mmol) of Nile Red, 11.0 g (4.33 mmol) of fluorinated phenylacetonitrile (2) represented by formula (17), and 50 ml of acetic anhydride were placed in a 100 ml pear-shaped flask. The solution in the pear-shaped flask was heated in a silicone oil bath to 135° C. and allowed to react for 1 hour. Acetic anhydride was distilled away with an evaporator and the residue was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 50 mg of violaceous solid was obtained. The melting point of the product was 250-252° C. An NMR chart of this product is shown in FIG. 5. The results of elemental analysis of this violaceous product are as follows. Based on these results, the obtained product was identified as the Nile Red luminescent compound represented by formula (18).

The results of elemental analysis

Found values: C: 68.89, H: 4.40, N: 5.43

Calculated values: C: 68.77, H: 4.38, N: 5.53

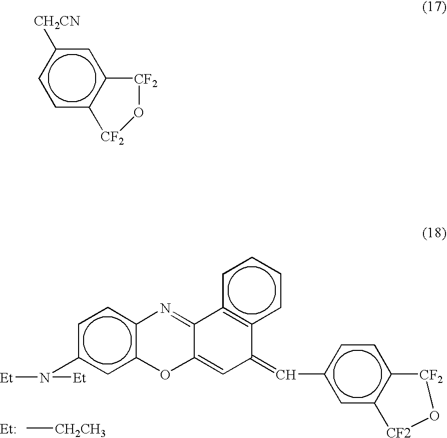

In a 5 ml graduated flask were placed 70.0 mg of polyvinyl carbazole, which will be abbreviated to PVK hereinafter, 29.7 mg of BND, and 0.3 mg of the Nile Red luminescent compound represented by formula (16). Dichloroethane was added to the mixture so that the total volume of the mixture was 5 ml. Then, the solution including the Nile Red luminescent compound was prepared. An EL element was made from this solution in the same manner as that of Working Example 7.

The highest luminance and the chromaticity of the EL element were measured with a Fast BM-7 measuring apparatus produced by TOPCON Corporation, with the voltage being raised gradually. When the voltage was 17 V and the current was 9.07 mA, the luminance was 1575 Cd/m² and the value on the x-axis in CIE chromaticity was 0.6552. When the voltage was 18 V and the current was 11.84 mA, the luminance was 1815 Cd/m² and the value on the x-axis in CIE chromaticity was 0.6563. When the voltage was 19 V and the current was 13.98 mA, the luminance was 1702 Cd/m² and the value on the x-axis in CIE chromaticity was 0.6559. When the voltage was 20 V and the current was 16.84 mA, the luminance was 1505 Cd/m² and the value on the x-axis in CIE chromaticity was 0.6517.

An ITO substrate was cleaned in the same manner as that of Example 7 and the cleaned ITO substrate was set in a vacuum metallizer. Under a reduced pressure of not more than 1×10⁻⁶ torr, N,N′-diphenyl-N,N-di(m-tolyl)-benzidine (TPD) was vapor-deposited on the substrate, which results in a film having a thickness of 60 nm. Then, a mixture of tris(8-quinolinate) aluminum (Alq3) and the Nile Red luminescent compound represented by formula (18), wherein the amount of the latter was 2 weight %, was vapor-deposited on the surface of the ITO-applied substrate, so that the thickness of the upper film was 31 nm. Finally, the aluminum electrode of 150 nm in thickness was formed on the surface of the upper film by vapor-deposition. Thus an EL element was prepared.

The luminance and the chromaticity of the EL element were measured in the same manner as those of Example 7. When the voltage was 27 V and the current was 15.37 mA, the luminance was 3660 Cd/m², and the value on the x-axis in CIE chromaticity was 0.6266.

### Working Example 18

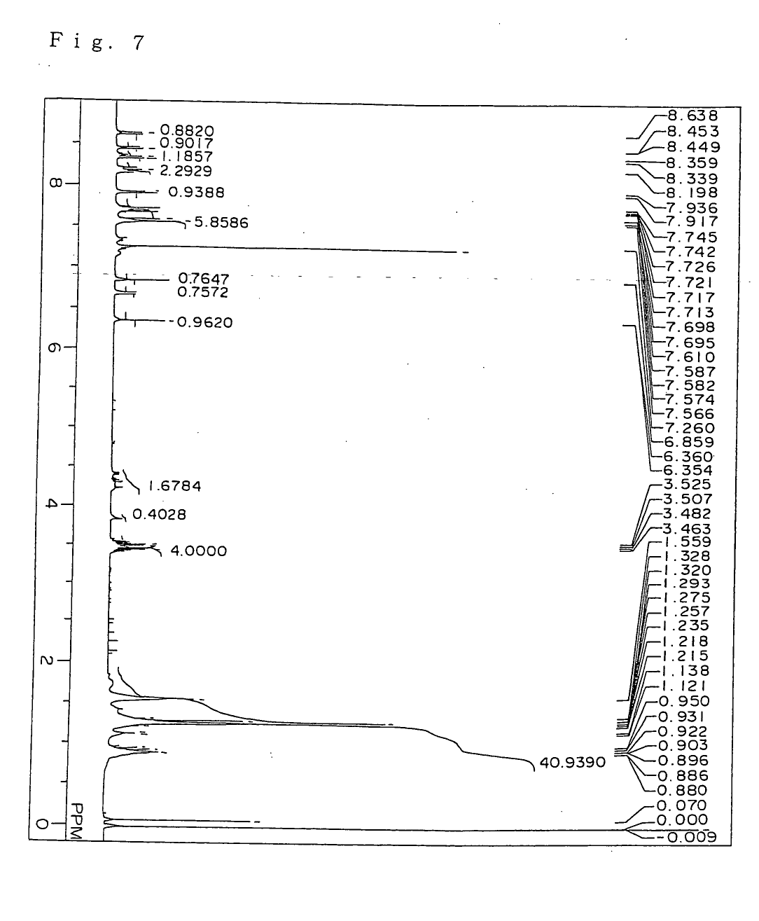

0.92 g (2.89 mmol) of Nile Red, 11.0 g (4.33 mmol) of fluorinated phenylacetonitrile (3) represented by formula (19), and 60 ml of acetic anhydride were placed in a 100 ml pear-shaped flask. The solution in the pear-shaped flask was heated in a silicone oil bath to 135° C. and allowed to react for 3.5 hours. Acetic anhydride was distilled away with an evaporator and the residue was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 10 mg of violaceous solid was obtained. An NMR chart of this product is shown in FIG. 7. The results of elemental analysis of this violaceous product are as follows. Based on these results, the obtained product was identified as the Nile Red luminescent compound represented by formula (20).

The results of elemental analysis

Found values: C: 75.19, H: 5.01, N: 5.43

Calculated values: C: 75.28, H: 4.94, N: 5.49

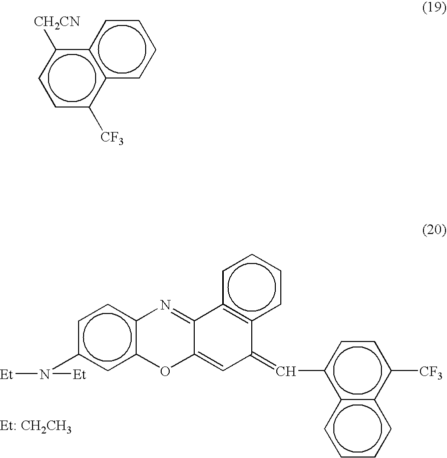

### Working Example 19

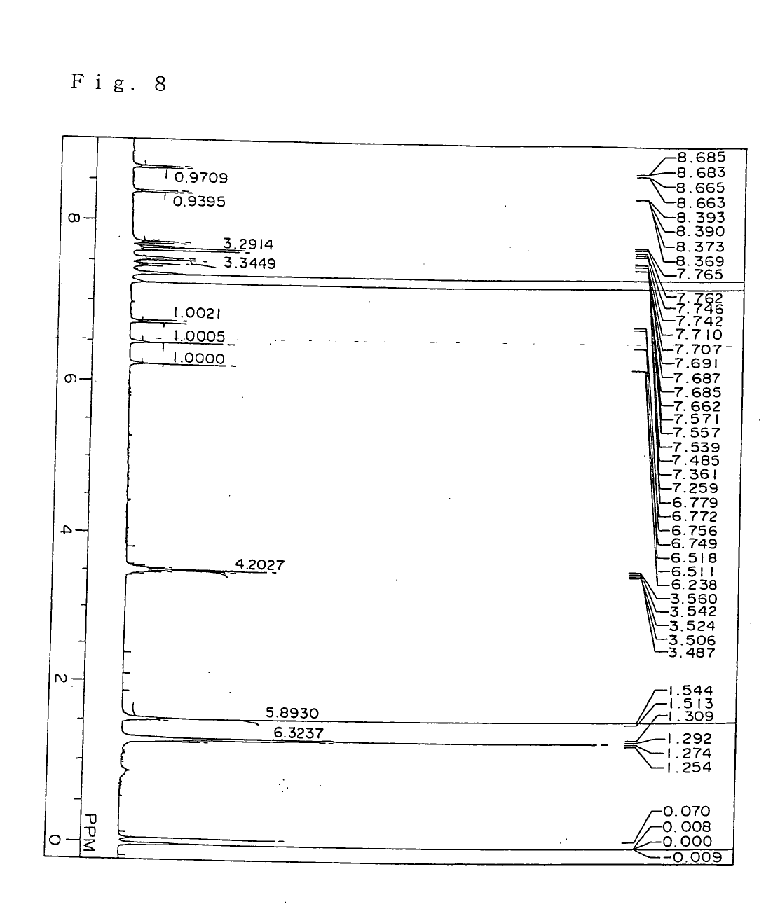

1.04 g (3.28 mmol) of Nile Red, 11.0 g (4.92 mmol) of 3-fluoro-4-(trifluoromethyl)phenylacetonitrile, and 50 ml of acetic anhydride were placed in a 100 ml pear-shaped flask. The solution in the pear-shaped flask was heated in a silicone oil bath to 135° C. and allowed to react for 2.5 hours. Acetic anhydride was distilled away with an evaporator and the residue was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 40 mg of violaceous solid was obtained. The melting point of the violaceous solid was 215-220° C. An NMR chart of this product is shown in FIG. 8. The results of elemental analysis of this violaceous product are as follows. Based on these results, the obtained product was identified as the Nile Red luminescent compound represented by formula (21).

The results of elemental analysis

Found values: C: 70.19, H: 4.77, N: 5.83

Calculated values: C: 70.29, H: 4.63, N: 5.85

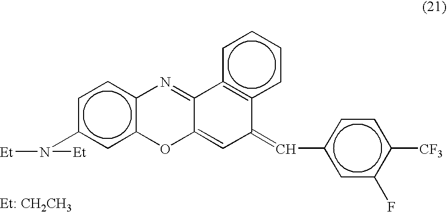

In a 5 ml graduated flask were placed 70.0 mg of polyvinyl carbazole, a product produced by Kanto Kagaku Co., Ltd., which polyvinyl carbazole will be abbreviated to PVK, 29.8 mg of BND, and 0.2 mg of the Nile Red luminescent compound represented by formula (20). Dichloroethane was added to the mixture so that the total volume of the mixture was 5 ml. An EL element was prepared from the obtained solution containing this Nile Red luminescent compound in the same way as that explained in Example 7 above.

The highest luminance and the chromaticity of the EL element were measured with a Fast BM-7 measuring apparatus produced by TOPCON Corporation, with the voltage being raised gradually. When the voltage was 16 V and the current was 6.16 mA, the luminance was 1087 Cd/m² and the value on the x-axis in CIE chromaticity was 0.6776. When the voltage was 17 V and the current was 9.32 mA, the luminance was 1444 Cd/m² and the value on the x-axis in CIE chromaticity was 0.6780. When the voltage was 18 V and the current was 12.71 mA, the luminance was 1622 Cd/m² and the value on the x-axis in CIE chromaticity was 0.6787. When the voltage was 19 V and the current was 15.73 mA, the luminance was 1455 Cd/m² and the value on the x-axis in CIE chromaticity was 0.6790. When the voltage was 20 V and the current was 18.28 mA, the luminance was 1120 Cd/m² and the value on the x-axis in CIE chromaticity was 0.6710.

An ITO substrate was cleaned in the same manner as that of Example 7 and the cleaned ITO substrate was set in a vacuum metallizer. Under a reduced pressure of not more than 1×10-6 torr, N,N′-diphenyl-N,N-di(m-tolyl)-benzidine (TPD) was vapor-deposited on the substrate, which results in a film having a thickness of 60 nm. Then, a mixture of tris(8-quinolinate) aluminum (Alq3) and the Nile Red luminescent compound represented by formula (20), wherein the amount of the latter was 1.7 weight %, was vapor-deposited on the surface of the ITO-applied substrate, so that the thickness of the upper film was 31 nm. Finally, the aluminum electrode of 150 nm in thickness was formed on the surface of the upper film by vapor-deposition. Thus an EL element was prepared.

The luminance and the chromaticity of the EL element were measured in the same manner as that of Example 7. When the voltage was 27 V and the current was 18.53 mA, the luminance was 6043 Cd/m², and the value on the x-axis in CIE chromaticity was 0.6326.

### Working Example 20

<Synthesis of 6-amino-3-(diisopropylamino)phenol>

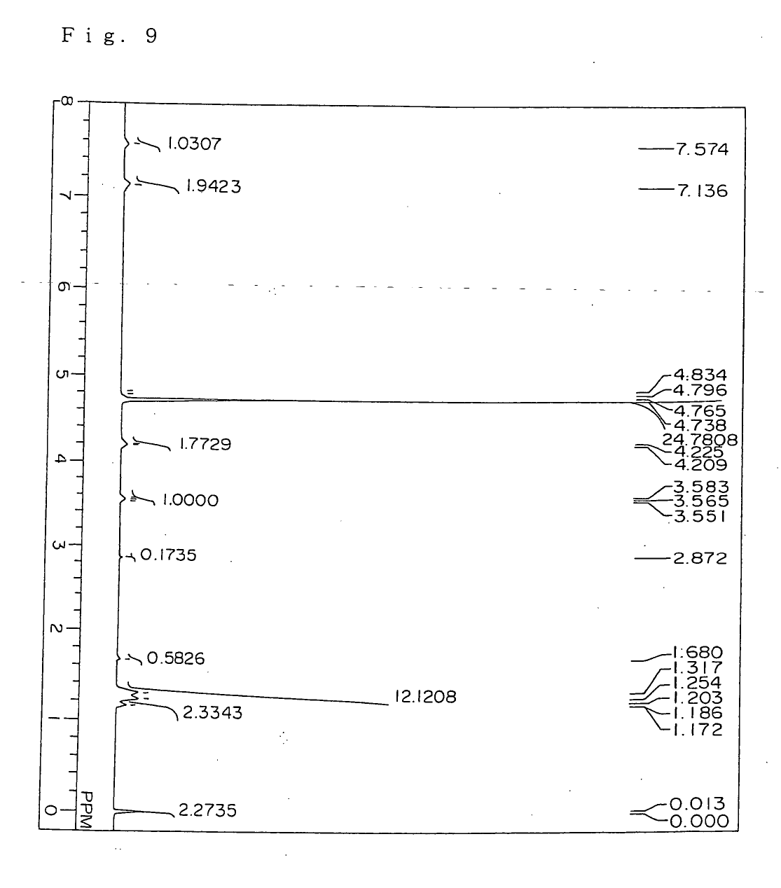

In a 500 ml three-necked flask, 26 g (115 mmol) of stannous chloride dihydrate and 28 ml of a concentrated hydrochloric acid were placed. The mixture was heated and boiled. 60 ml of an acetic acid solution of 5.5 g (23.1 mmol) of 5-(diisopropylamino)-2-nitrophenol was dropped in the mixture. After the completion of the dropping, the obtained mixture was allowed to react at the reflux temperature for 1 hour. Then, the mixture was cooled to the room temperature. Water and acetic acid were completely distilled away. The residue was dissolved in 200 ml of water and the pH of the aqueous solution was adjusted to 3-4 with a 5% aqueous solution of sodium hydroxide. The precipitated solid was filtered out and the filtrate was concentrated. The precipitated solid was washed with ether and vacuum-dried, which resulted in 16.9 g of beige solid. This solid included salt. An NMR chart of this beige solid is shown in FIG. 9. This solid was identified as 6-amino-3-(diisopropylamino)phenol.

<Synthesis of a Nile Red Compound Derivative>

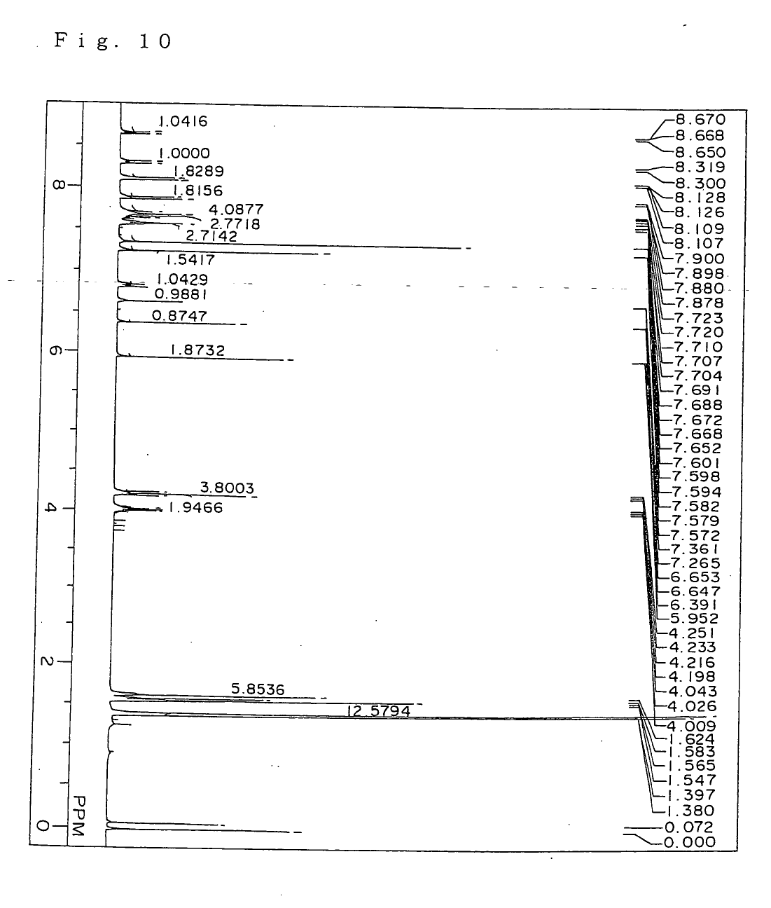

In a 500 ml pear-shaped flask were placed 16.9 g of 6-amino-3-(diisopropylamino)phenol, 4.0 g (23.1 mmol) of 2-hydroxy-1,4-naphtoquinone, and 150 ml of ethanol. The mixture was allowed to react in the presence of boiling tips under reflux for 21 hours. The reaction liquid was concentrated with an evaporator, and alkalized with 200 ml of a 10% aqueous solution of sodium hydroxide. This alkaline liquid was extracted with chloroform and washed with water. The extract was dried with anhydrous sodium sulfate and then chloroform was distilled off. The treated extract was purified by a column chromatography employing silica gel. 0.50 g of the aimed Nile Red compound derivative was obtained. An NMR chart of this Nile Red compound derivative is shown in FIG. 10.

<Synthesis of a Nile Red Luminescent Compound>

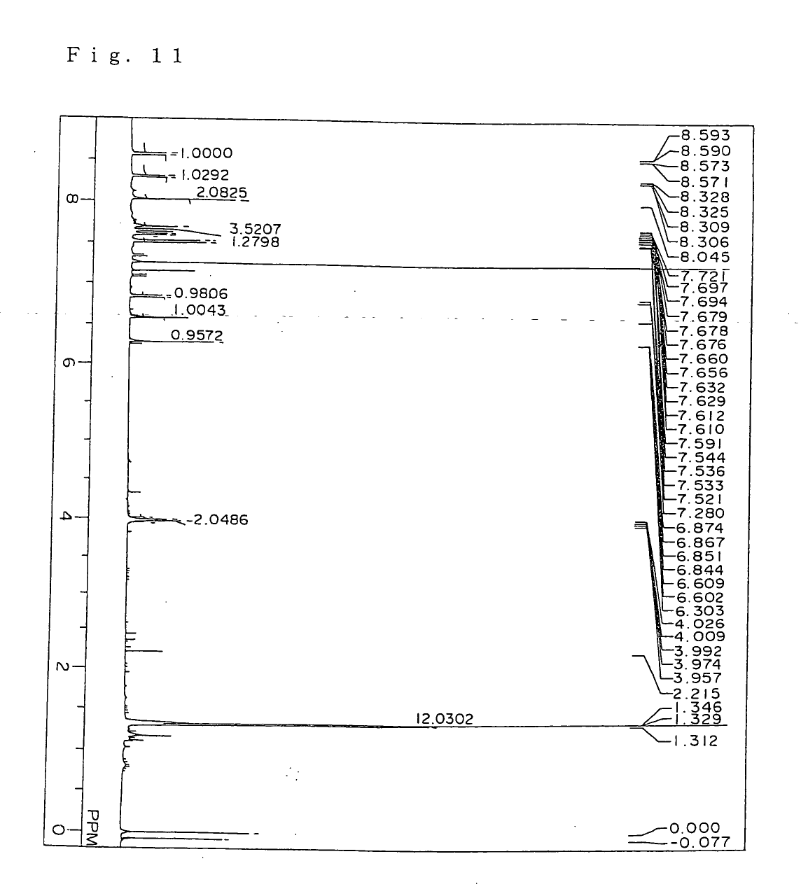

0.46 g (1.33 mmol) of the Nile-red compound derivative, 0.50 g (1.99 mmol) of 3,5-bis(trifluoromethyl)phenylacetonitrile, and 50 ml of acetic anhydride were placed in a 100 ml pear-shaped flask. The solution in the pear-shaped flask was heated in a silicone oil bath to 135° C. and allowed to react for 2 hours. Acetic anhydride was distilled off with an evaporator and the residue was dissolved in chloroform. This chloroform solution was washed with a 5% aqueous solution of sodium hydroxide and then with water. After the addition of sodium sulfate, the solution was allowed to stand for 30 minutes to be dried. The dried solution was concentrated with an evaporator. The obtained solid was purified by a column chromatography that used silica gel and benzene. 14 mg of violaceous solid was obtained. The melting point of the violaceous solid was 183-185° C. An NMR chart of this product is shown in FIG. 11. The results of elemental analysis of this violaceous product are as follows. Based on these results, the obtained product was identified as the Nile Red luminescent compound represented by formula (22).

The results of elemental analysis

Found values: C: 67.02, H: 4.77, N: 5.13

Calculated values: C: 66.90, H: 4.71, N: 5.03

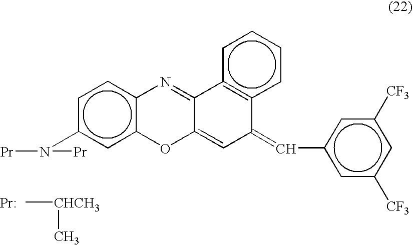

### INDUSTRIAL APPLICABILITY

This invention can provide a novel luminescent compound capable of emitting at a high luminance a light of which color is nearly crimson, and of enduring heat and light.

This invention can also provide a novel Nile Red luminescent compound, from which a luminescent element emitting white light can be prepared.

Further, this invention can provide a process for easily producing the Nile Red luminescent compound.

Still further, this invention can provide an EL element that emits at a high luminance a light of which color is nearly crimson.

We claim:

1. A Nile Red luminescent compound emitting red light comprising a compound represented by formula (1): 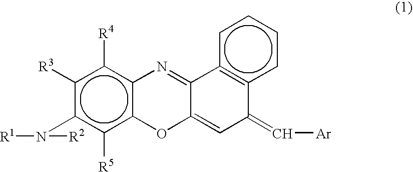
wherein R¹ is a lower alkyl having 1-5 carbon atoms, or forms —CH₂CH₂—CR⁶R⁷— together with R³(wherein the carbon atom of —CR⁶R⁷-moiety is bound to the benzene moiety of formula (1), each of R⁶ and R⁷ is hydrogen atom or a lower alkyl having 1-5 carbon atoms, and R⁶ and R⁷ may be the same or different from each other); R² is a lower alkyl having 1-5 carbon atoms, or forms —CH₂CH₂—CR⁸R⁹— together with R⁵ (wherein the carbon atom of —CR⁸R⁹— moiety is bound to the benzene moiety of formula (1), each of R⁸ and R⁹ is hydrogen atom or a lower alkyl having 1-5 carbon atoms, and R⁸ and R⁹ may be the same or different from each other); R³ is hydrogen atom, forms —CH₂CH₂—CR⁶R⁷— with R¹, or forms with R⁴ a naphthalene ring including as a part thereof the benzene moiety of formula (1); R⁴ is hydrogen atom, or forms with R³ a naphthalene ring including as a part thereof the benzene moiety of formula (1); R⁵ is hydrogen atom, or forms —CH₂CH₂—CR⁸R⁹— with R²; and Ar means one of the following general formulae (2), (4) and (5): 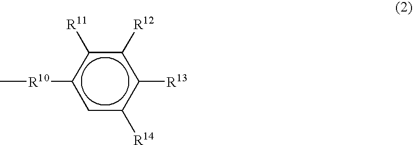
wherein R¹⁰ is carbon atom or methylene group; R¹¹ is hydrogen atom, or forms —CF₂—O—CF₂— with R¹²; R¹² is fluorine atom, cyano group or a lower alkyl having 1-5 carbon atoms and at least one fluorine atom, forms —CF₂—O—CF₂— with R¹¹, or forms —CF₂—O—CF₂— with R¹³; R¹³ is hydrogen atom, cyano group, fluorine atom or a lower alkyl having 1-5 carbon atoms and at least one fluorine atom, forms —CF₂—O—CF₂— with R¹², or is a group represented by general formula (3); and R¹⁴ is hydrogen atom when R¹³ is not hydrogen atom, or a lower alkyl having 1-5 carbon atoms and at least one fluorine atom when R¹³ is hydrogen atom, 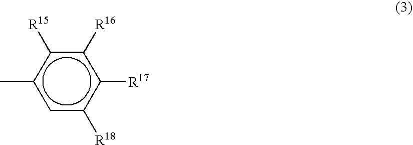
wherein R¹⁵ is hydrogen atom, or forms —CF₂—O—CF₂— with R¹⁶; R¹⁶ is fluorine atom, cyano group or a lower alkyl having 1-5 carbon atoms and at least one fluorine atom, forms —CF₂—O—CF₂— with R¹⁵, or forms —CF₂—O—CF₂— with R¹⁷; R¹⁷ is hydrogen atom, cyano group, fluorine atom or a lower alkyl having 1-5 carbon atoms and at least one fluorine atom, or forms —CF₂—O—CF₂— with R¹⁶; and R¹⁸ is hydrogen atom when R¹⁷ is not hydrogen atom, or a lower alkyl having 1-5 carbon atoms and at least one fluorine atom when R¹⁷ is hydrogen atom, 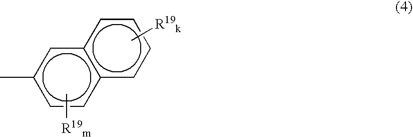
wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other, 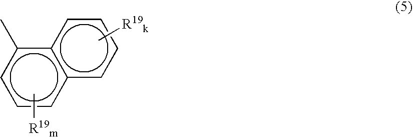
wherein R¹⁹ is fluorine atom, cyano group, or a lower alkyl having 1-5 carbon atoms and at least one fluorine atom; k is an integer of 1-4, m is an integer of 1-3, and all of the R¹⁹ groups may be the same or different from each other.

2. A process for producing the Nile Red luminescent compound emitting red light represented by formula (1), comprising reacting a Nile Red pigment represented by general formula (6): 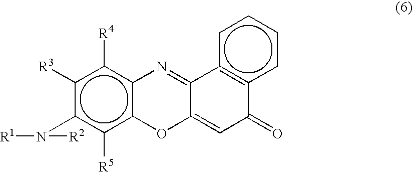
wherein R¹, R², R³, R⁴ and R⁵ are respectively the same as those defined in the explanation of formula (1), with an electron attractive aromatic acetonitrile represented by general formula (7):
NC—CH₂—Ar (7)
wherein Ar is the same as that defined in the explanation of formula (1).

3. A luminescent element comprising a pair of electrodes and a luminescent layer including the Nile Red luminescent compound represented by formula (1) between the electrodes.

4. The luminescent element as claimed in claim 3, further comprising a hole-transporting layer between the luminescent layer and the cathode that is one of the electrodes

5. The luminescent element as claimed in claim 4, wherein the luminescent layer includes the Nile Red luminescent compound and a host pigment.

6. The luminescent element as claimed in claim 4 or 5, wherein the luminescent layer and the hole-transporting layer are formed by deposition.

7. The luminescent element as claimed in claim 3, wherein the luminescent layer includes the Nile Red luminescent compound, an electron-transporting substance and a hole-transporting high polymer.

8. The luminescent element as claimed in claim 7, wherein the luminescent layer is formed through coating.
